(19) United States
(12) Patent Application Publication
(10) Pub. No.: US 2007/0073172 A1
(43) Pub. Date: Mar. 29, 2007

(54) Stable pressure release device for electronic sphygmomanometer

(75) Inventors: Paul Yang, Chung Ho City (TW); Shan-Yi Yu, Zhonghe City (TW); Hsin-Yi Pai, Caotun Town (TW)

Correspondence Address: BACON & THOMAS, PLLC, 625 SLATERS LANE, FOURTH FLOOR, ALEXANDRIA, VA, 22314, US

(73) Assignee: HEALTH & LIFE CO., LTD., CHUNG HO CITY (TW)

(21) Appl. No.: 11/235,319

(22) Filed: Sep. 27, 2005

(51) Int. Cl. A61B 5/02

(52) U.S. Cl. 600/490

(57) ABSTRACT

A stable pressure release device for electronic sphygmomanometer includes a casing and a multi-passage interconnecting pipe on the casing. An end of the interconnecting pipe is connected to a cuff, a pump, a pulse detector, and a low airflow valve. The low airflow valve includes an air passage which has a piston made of a porous material, so that when the electronic sphygmomanometer is used, the pump pressurizes and sends air into the interconnecting pipe. After the cuff is filled up with air, the air in the cuff is released in a low airflow steadily to the outside through the small even air holes of the piston. Therefore, the pulse sensor can accurately detect a pulse signal of the blood pressure, since the airflow is small and stable. The piston will not be exhausted easily and the airflow is steady, and thus errors will not result easily during the detection.

### BACKGROUND OF THE INVENTION

1. Field of the Invention

The present invention relates to a stable pressure release device for electronic sphygmomanometer, and more particularly to a piston made of a porous material that uses small and even air holes of the porous material to steadily release a small quantity of airflow, so as to detect the pulse signal of our blood pressure more accurately.

2. Description of the Related Art

Referring to FIG. 1 for a prior art electronic sphygmomanometer, its related electronic components are installed on a circuit board 110, and a casing 100 wraps the circuit board 110 therein, and the circuit board 110 includes an LCD screen 120 protruded from the surface of the casing 100 for displaying the reading of measured blood pressures, and the casing 100 includes a multi-channel interconnecting pipe 130, and the interconnecting pipe 130 is connected to a connector 131, and the connector 131 is connected to a cuff (not shown in the figure), and the cuff is worn around the arm or the wrist of a user. The interconnecting pipe 130 is interconnected to a low airflow valve 132, a pulse sensor 133, a pump 134 and a high airflow valve 135, wherein the pulse sensor 133 is installed onto a circuit board 110 for detecting the pulse of the blood pressure of a human body as shown in FIG. 2. The low airflow valve 132 includes a containing space 1321 corresponding to an opening of the interconnecting pipe 130 and a hollow silicon (rubber) tube 1322 is disposed in the containing space 1321, and an end of the silicon (rubber) tube 1322 is closed, and the corresponding end has an opening. Further, the silicon (rubber) tube 1322 has a lengthwise crevice 1323 at its surface, and the lengthwise crevice 1323 is interconnected with the air in the containing space 1321 and the silicon (rubber) tube 1322, and the silicon (rubber) tube 1322 includes a screw cylinder 1324 embedded in an opening end, and the screw cylinder 1324 includes a penetrating air passage 13241 therein. Further, the penetrating air passage 13241 is interconnected with the open end of the silicon (rubber) tube 1322, and the screw cylinder 1324 is secured by a screw nut 1325. The screw nut 1325 is embedded into the containing space 1321, so that when the electronic sphygmomanometer is operated, the pump 134 pressurizes and sends the air into the cuff through the interconnecting pipe 130 until the air pressure reaches a maximum set value. The screw cylinder 1324 is turned with respect to the screw nut 1325 to move the silicon (rubber) tube 1322 originally pressed on the air passage 1326 up or down, so that the air in the cuff passes through the interconnecting pipe 130 and the air passage 1326, and enters into the silicon (rubber) tube 1322 through the crevice 1323 of the silicon (rubber) tube 1322. The penetrating air passage 13241 in the screw cylinder 1324 is used to flow the air out. By then, the high airflow valve 135 is closed, and the pulse sensor 133 detects the pulse of high/low blood pressure by slowly releasing the air, and the detected high/low blood pulse data is sent to the circuit board 110 for data processing, and the measured result is sent to and displayed on the LCD screen 120, and then the high airflow valve 135 is turned on to quickly discharge the remained air from the high airflow valve 135.

However, the low airflow valve 132 is generally made of silicon (rubber) material, and has an elastic exhaustion and thus will be deformed or become fragile after being used for some time. Therefore, the airflow entering into the containing space 1321 cannot be maintained steady, and thus causing an imprecise measurement.

Since the discharged airflow can be adjusted by changing the distance between the screw cylinder 1324 and the screw nut 1325 as shown in FIG. 2, and the threads in the screw cylinder 1324 and the screw nut 1325 are manufactured in a way that the distance between the threads can approach a certain precision, but it cannot be adjusted to a micro distance. The lengthwise crevice 1323 of the silicon (rubber) tube 1322 is cut by a mechanical method, and thus the airflow cannot be discharged in small quantity, and the pressure of the outgoing airflow is inconsistent, and thus the air pressure for each measurement cannot be reduced. For example, the airflow flowing into the containing space 1321 each time is 2-5 mmHg/second, and thus the air pressures measured by the pulse sensor 133 per second cannot be lowered and the interval cannot be shortened. As a result, the measurement is not very accurate.

### SUMMARY OF THE INVENTION

In view of the shortcomings of the prior art, the inventor of the present invention aimed at the problem and tried to find a way of overcoming the shortcomings of the prior art and conducted extensive researches and experiments to find a feasible solution, and finally invented a stable pressure release device for electronic sphygmomanometer in accordance with the present invention.

Therefore, it is a primary object of the present invention to overcome the shortcomings of the prior art by providing a stable pressure release device for electronic sphygmomanometer that comprises a casing, and an interconnecting pipe disposed on the casing, and one end of the interconnecting pipe is coupled with a cuff, a pump, a pulse sensor, and a low airflow valve, wherein the low airflow valve includes an air passage, and a piston made of a porous material and disposed in the air passage. When the electronic sphygmomanometer is used, the pump pressurizes and sends the air into the interconnecting pipe. After the cuff is filled up with air, the small and even air holes of the piston can steadily discharge the air from the cuff to the outside in a small quantity through the air holes. The discharged airflow is small and stable, so that the pulse sensor can detect a smaller change of the measured air pressure at a specific time than the prior art device and obtains a more accurate measurement of blood pressure. Since the piston will not get elastically exhausted easily, the measurement can be more stable and error will not occur so easily.

Another object of the present invention is to provide a stable pressure release device for electronic sphygmomanometer that includes a high airflow valve connected to the interconnecting pipe for quickly discharging the air to the outside.

Another further object of the present invention is to provide a stable pressure release device for electronic sphygmomanometer that includes a piston made of a precision ceramic material.

### BRIEF DESCRIPTION OF THE DRAWINGS

To make it easier for our examiner to understand the objective, shape, assembly, structure, characteristics and performance of the present invention, the following embodiments accompanied with the related drawings are described in details.

FIG. 1 is a schematic view of a prior art device;

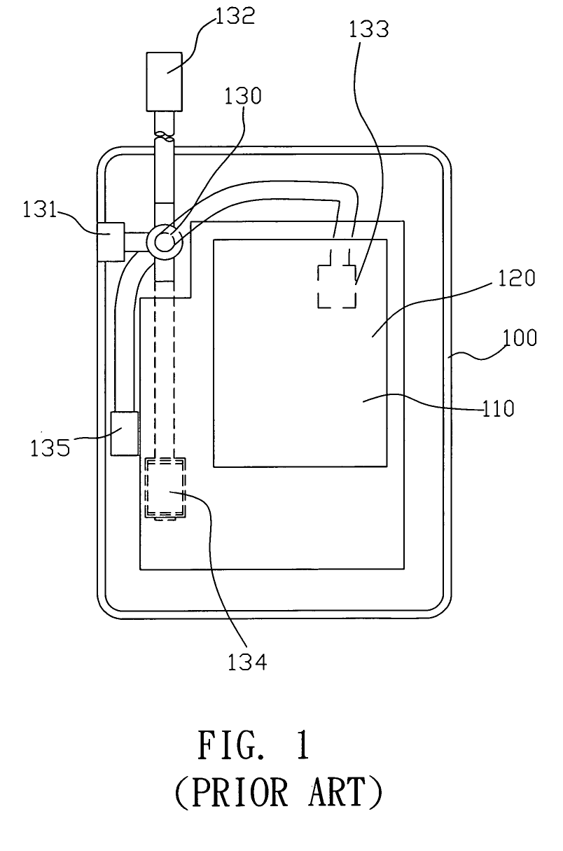

FIG. 2 is a cross-sectional view of a low airflow valve of a prior art;

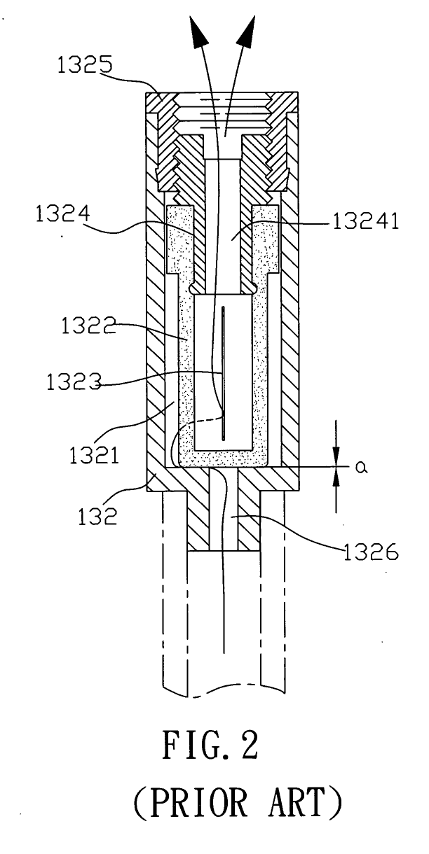

FIG. 3 is a view of dissembled parts of a low airflow valve of the invention;

FIG. 4 is a cross-sectional view of a low airflow valve of the invention;

FIG. 5 is a view of disassembled parts of a low airflow valve according to another preferred embodiment of the invention;

FIG. 6 is a cross-sectional view of a low airflow valve according to another preferred embodiment of the invention; and

FIG. 7 is a schematic view of the use of the invention.

### DETAILED DESCRIPTION OF THE PREFERRED EMBODIMENTS

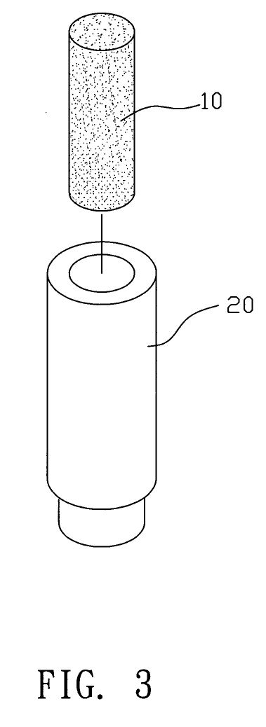

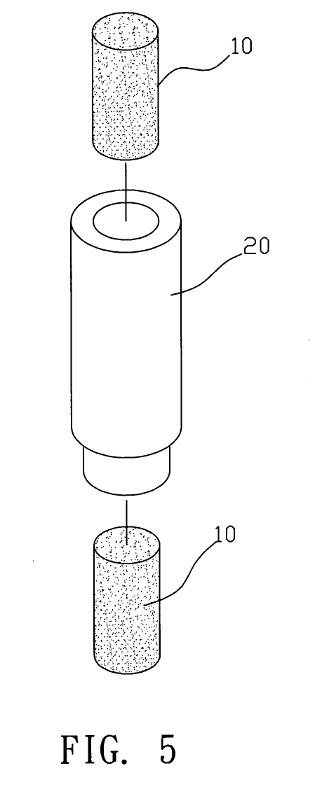

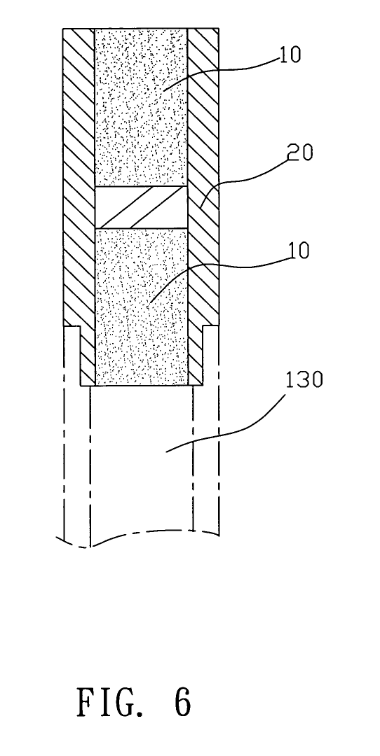

Referring to FIGS. 3, 4, and 7 for the stable pressure release device for electronic sphygmomanometer of the present invention, the device comprises a casing 100, a circuit board 110 wrapped inside the casing 100, an LCD screen 120 connected to the circuit board 110 and protruded from the surface of the casing 100 for displaying the measured high/low blood pressure values, and the casing 100 includes a multi-channel interconnecting pipe 130, and the interconnecting pipe 130 includes a connector 131 coupled thereon, and the connector 131 is coupled with a cuff (not shown in the figure), and the interconnecting pipe 130 is connected to a pulse sensor 133, a pump 134, and a high airflow valve 135, wherein the pulse sensor 133 is installed on the circuit board 110 for detecting the pulse of the blood pressure and measuring the high and low blood pressures. Further, the interconnecting pipe 130 has a piston 10 installed at an end where the foregoing devices are not installed, and the piston 10 is made of a porous material which is a precision ceramic material in this preferred embodiment. However, the persons skilled in the art can use a foam material or a nano material or equivalents to substitute the precision ceramic material. The piston 10 passes through a hollow support member 20, and the hollow support member 20 is connected to an end of the interconnecting pipe 130 where the connector 131, pulse sensor 133, pump 134 and high airflow valve 135 are not installed, or directly passes through that end. Further, there could be one or two pistons (as shown in FIGS. 5 and 6).

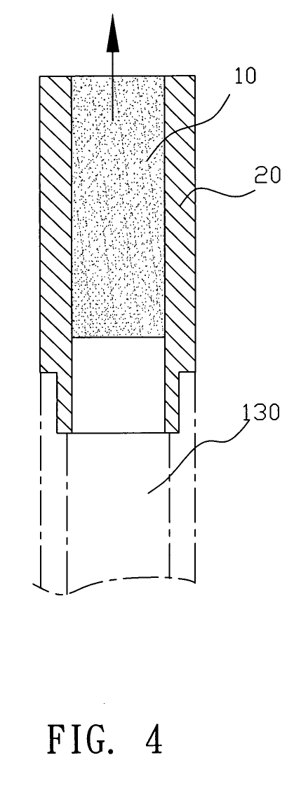

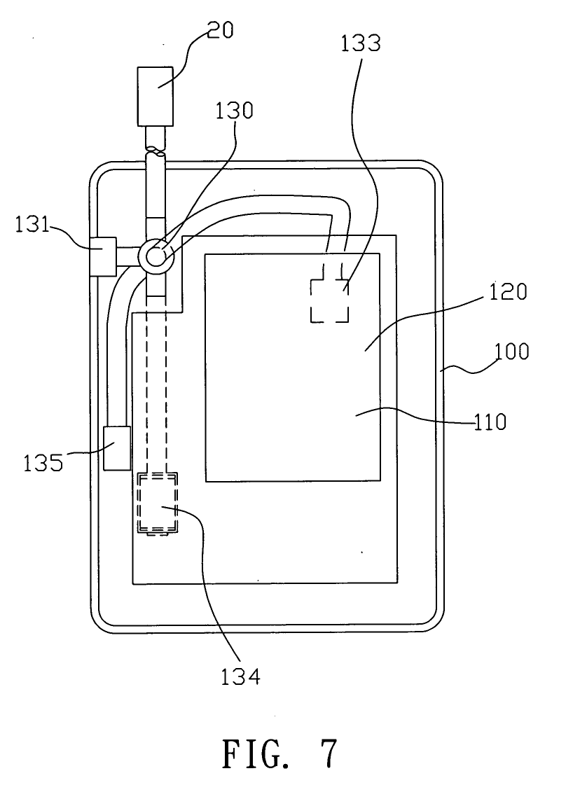

Referring to FIGS. 4 and 7 for the use of the electronic sphygmomanometer, the pump 134 sends air into the cuff through the interconnecting pipe 130, and the cuff is sheathed onto an arm or a wrist of a user. By then, the high airflow valve 135 is closed. After the air pressure reaches a maximum set value, the air is released from the small and even air holes of the piston 10 to the outside steadily with a small quantity, such that the pulse sensor 133 can measure the blood pressure more accurately, because the airflow is released steadily when the blood pressure is measured. Since the piston 10 will not get elastically exhausted easily or the airflow remains stable, therefore error will not be occurred easily. The pulse sensor 133 detects the pulse of the high/low blood pressure by slowly releasing the air, and the detected high/low blood pressure pulse data is sent to the circuit board 110 for data processing, and the measured result is sent to and displayed on the LCD screen 120, Then, the high airflow valve 135 is turned on to quickly discharge the remained air to the outside from the high airflow valve 135.

In summation of the above description, the present invention herein enhances the performance and overcomes the shortcoming of the prior art, and further complies with the patent application requirements.

While the invention has been described by way of example and in terms of a preferred embodiment, it is to be understood that the invention is not limited thereto. To the contrary, it is intended to cover various modifications and similar arrangements and procedures, and the scope of the appended claims therefore should be accorded the broadest interpretation so as to encompass all such modifications and similar arrangements and procedures.

What is claimed is:

1. A stable pressure release device for electronic sphygmomanometer, having a multi-passage interconnecting pipe, and an end of said interconnecting pipe being coupled to a cuff, a pump, and a pulse sensor, characterized in that:
said interconnecting pipe comprises a piston disposed at an end of said interconnecting pipe, and said piston is made of a porous material, so that when said electronic sphygmomanometer is used, said pump pressurizes and sends air into said interconnecting pipe, and after said cuff is filled up with air, the air in said cuff is discharged steadily in a small airflow to the outside through said small and even air holes, such that said pulse sensor can obtain a more accurate measurement, since said piston will not be exhausted easily and the airflow is steadily, and thus errors will not result easily.

2. The stable pressure release device for electronic sphygmomanometer of claim 1, wherein said piston is made of a precision ceramic material.

3. The stable pressure release device for electronic sphygmomanometer of claim 1, wherein said piston passes through a hollow supporting member.

4. The stable pressure release device for electronic sphygmomanometer of claim 1, comprising one piston.

5. The stable pressure release device for electronic sphygmomanometer of claim 1, comprising two pistons.
